(19) United States
(12) Patent Application Publication
(10) Pub. No.: US 2020/0397857 A1
(43) Pub. Date: Dec. 24, 2020

(54) METHODS AND COMPOSITIONS FOR PROMOTING OPC DIFFERENTIATION AND REMYELINATION USING RECEPTOR ASSOCIATED PROTEIN (RAP)

(71) Applicant: Novoron Bioscience, Inc., San Diego, CA (US)

(72) Inventor: Travis STILES, San Diego, CA (US)

(21) Appl. No.: 16/867,058

(22) Filed: May 5, 2020

Related U.S. Application Data
(63) Continuation of application No. 16/138,743, filed on Sep. 21, 2018, now abandoned.
Application No. 16/138,743 is a continuation of application No. PCT/US17/23481, filed on Mar. 21, 2017.
(60) Provisional application No. 62/400,886, filed on Sep. 28, 2016.
(60) Provisional application No. 62/311,095, filed on Mar. 21, 2016.

(51) Int. Cl. A61K 38/17; C12N 15/86; C07K 14/47; C07K 14/705; C12N 5/079; A61P 25/00; A61K 9/00

(52) U.S. Cl. CPC A61K 38/177; C12N 15/86; C07K 14/47; C07K 14/70596; C12N 2501/998; A61P 25/00; A61K 9/0019; C12N 2501/395; C12N 5/0622

(57) ABSTRACT

The present disclosure relates to methods and compositions using RAP, a derivative of RAP, a variant of RAP, or a fragment of RAP to inhibit LRP1, a myelin debris receptor. The methods and compositions involve increasing, promoting, restoring, and/or enhancing differentiation of oligodendrocyte progenitor cells, myelin protein expression, mature oligodendrocyte marker expression, and/or myelination. The methods and compositions disclosed herein inhibit or block pathological activation of RhoA in OPCs. The methods and compositions also involve alleviating one or more symptoms of MS and treating MS, including slowing or stopping MS progression.

### CROSS-REFERENCE TO RELATED APPLICATIONS

This application is a continuation of U.S. application Ser. No. 16/138,743, filed Sep. 21, 2018, which is a continuation of International Application PCT/US2017/023481, filed on Mar. 21, 2017, and claims the benefit of and priority to U.S. Provisional Application 62/311,095 filed Mar. 21, 2016, and U.S. Provisional Application 62/400,886 filed on Sep. 28, 2016, the entire contents of each of which are herein incorporated by reference in their entireties.

### FIELD OF THE DISCLOSURE

The present disclosure relates to receptor associated protein (RAP) as a therapeutic for promoting myelination, including remyelination to address symptoms of multiple sclerosis.

### BACKGROUND

Multiple sclerosis (MS) is a neurodegenerative disease in which myelin of the central nervous system (CNS) is destroyed by a self-reactive immune response that is accompanied by death of the oligodendrocytes, the myelinating cells of the CNS. Relapsing-remitting MS (RRMS) is the most common form of MS, affecting more than 80% of MS patients. RRMS has two phases: the auto-inflammatory episode (inflammatory phase), in which the immune system is actively destroying myelin, alternating with a remission phase. The majority of RRMS cases eventually progress into secondary progressive MS (SPMS), with greater than 50% of newly diagnosed RRMS patients progressing to SPMS within 10 years.

During the inflammatory phase of MS, immune cells invade the brain parenchyma and actively destroy oligodendrocytes, which disrupt the integrity of the myelin sheath. The loss of myelin sheath leaves axons exposed, making them more vulnerable to stress generated by the inflammatory response, such as the reactive oxygen species released by macrophages and microglia.

Chronic, and/or repeated bouts of, demyelination is a major cause of neuronal dysfunction and neurodegeneration in MS. However, currently approved therapies for MS are aimed at reducing the severity and frequency of MS attacks and do not address the need for stimulation of remyelination. The lack of agents capable of mitigating or resolving the irreversible damage caused by immune-mediated demyelinating lesions represents a fundamental gap in our ability to overcome disability and prevent death caused by this disease. Thus, there remains a need for other therapeutic methods and compositions for treatment of MS that are directed to the stimulation of remyelination.

### SUMMARY

The present disclosure relates to methods and compositions using RAP, a derivative of RAP, a variant of RAP, or a fragment of RAP to inhibit low-density lipoprotein receptor-related protein-1 (LRP1), a myelin debris receptor. The methods and compositions involve increasing, promoting, restoring, and/or enhancing differentiation of oligodendrocyte progenitor cells (OPCs), myelin protein expression, mature oligodendrocyte marker expression, and/or myelination. The methods and compositions disclosed herein inhibit or block pathological activation of ras homolog gene family, member A (RhoA) in OPCs. The methods and compositions also involve alleviating one or more symptoms of MS and treating MS, including slowing or stopping MS progression.

### BRIEF DESCRIPTION OF THE DRAWINGS

FIGS. 1A and 1B show mRNA expression of myelin basic protein (MBP) in the presence of a RAP derivative.

FIG. 2 shows the viability of OPCs in response to increasing doses of a RAP derivative.

FIG. 3 is a graph showing the effect of in vivo infusion of RAP into the lesion site of an injured spinal cord on RhoA activation.

### DETAILED DESCRIPTION

The present methods and compositions are based, in part, on administering receptor associated protein (RAP), or a derivative, variant, or fragment thereof, which acts as an antagonist to the myelin receptor low-density lipoprotein receptor-related protein-1 (LRP1) to block pathological activation of RhoA, a small GTPase protein of the Rho family, in OPCs. Blocking activation of RhoA permits OPC differentiation into mature oligodendrocytes which can carry out remyelination. Administering RAP, or a derivative, variant, or fragment thereof, can have upstream and downstream signaling effects from its interaction with LRP1. This makes RAP, or a derivative, variant, or fragment thereof, useful in various methods including methods for increasing, promoting, restoring, or enhancing myelin protein expression, expression of one or more mature oligodendrocyte markers, and myelination (including remyelination), and methods for alleviating one or more symptoms of MS and treating MS.

Remyelination is an endogenous repair mechanism that readily occurs in the healthy brain and spinal cord whereby new myelin is produced in response to myelin damage via recruitment, proliferation, and differentiation of oligodendrocyte precursor cells (OPCs) into myelin-forming oligodendrocytes, and is necessary to protect axons from further damage. Remyelination is also referred to as “myelination,” “lesion healing,” “neuronal repair,” and similar terms.

The CNS contains a large population of OPCs that have the potential to differentiate into mature oligodendrocytes and remyelinate denuded axons. Following a non-MS related episode of demyelination, OPCs are readily recruited into lesion sites where they proliferate and subsequently mature into myelinating oligodendrocytes. Although OPCs are efficiently recruited into MS lesions in patients, OPC differentiation into mature oligodendrocytes and subsequent remyelination is inhibited by myelin debris, which can linger in the area of demyelination. The remnants of disorganized myelin from previously destroyed oligodendrocytes are the most potent inhibitors of OPC maturation. This lesion persistence often worsens as the disease progresses and is largely responsible for the progressive disability, and death, caused by MS.

Identification of OPC receptors that mediate the suppressive signaling caused by myelin debris represents another potent therapeutic target for the enhancement of remyelination in MS. Low-density lipoprotein receptor-related protein-1 (LRP1) is a receptor for myelin debris that is responsible for myelin-mediated inhibition of myelin repair. Mature oligodendrocytes residing in the brain express negligible levels of LRP1, but OPCs are one of the most abundant LRP1 expressing cell types in the CNS. As such, LRP1 is as a high-value therapeutic target in the treatment of MS.

LRP1 is a multi-functional cell surface receptor involved in phagocytosis and cell signaling. It is a type-1 transmembrane receptor that binds over forty structurally and functionally distinct ligands, mediating their endocytosis and delivery to lysosomes. LRP1 also regulates cell-signaling by serving as a co-receptor or by regulating the trafficking of other receptors, such as uPAR, TNFR1, and PDGF receptor. The function of LRP1 in conjunction with other cell-signaling receptors explains the activity of LRP1 in regulation of inflammation, atherogenesis, and cell growth.

LRP1 is a central mediator of the pathologic hyperactivation of RhoA in neuronal cell types. This pathological hyperactivation of RhoA in neurons after CNS damage such as spinal cord injury is the necessary and sufficient signal for regenerative failure. The centrality of LRP1 in this process is related to its role in facilitating the pathological activation of RhoA, the biological signal responsible for regenerative suppression. Hyperactivation of RhoA has been demonstrated to be central to the lesion persistence in MS. Hyperactivation and subsequent suppression of OPC maturation has also been shown to be caused by factors within the lesion, such as molecules contained in disorganized CNS myelin and components of extracellular matrix proteins deposited in response to gliosis. Suppression of OPC differentiation in MS leads to failed remyelination and tissue repair in this disease. Lesion persistence often worsens as the disease progresses and is responsible for the progressive disability and death caused by the disease.

The failure of lesions to remyelinate in MS is due to pathological RhoA activation in OPCs. However, many agents designed to target this pathway do so indiscriminately via pan inhibition of RhoA, or its downstream effector Rho-associated kinase (ROCK), which can impair important basal cellular functions and contribute to risk of toxicity.

Inhibition of LRP1 expression in primary OPCs overcomes myelin inhibition of differentiation in vitro, and results in increased expression of myelin proteins. Importantly, LRP1 does not modulate non-pathological activators of RhoA activity that are responsible for endogenous cellular functions. This is a dramatic improvement over pan-Rho/ROCK targeted approaches. As LRP1 is a receptor for myelin debris and is important for myelin-mediated OPC suppression of differentiation, LRP1 is a therapeutic target for promoting OPC differentiation, enhancing myelin protein expression, promoting myelination, blocking pathological RhoA activation, and treating MS.

Receptor associated protein (RAP) is a highly flexible protein, having three domains (referred to as domains 1, 2, and 3; RAP-D1, RAP-D2, RAP-D3; and D1, D2, and D3) of three helical bundles that are loosely joined by flexible linkers. SEQ ID NO:1 is a rat RAP amino acid sequence. SEQ ID NO:2 is a human RAP amino acid sequence. SEQ ID NO:3 is a human RAP nucleic acid sequence (GenBank Accession No. NM 002337.2). SEQ ID NO:4 is a human RAP amino acid sequence (GenBank Accession No. NP 002328.1). SEQ ID NO:5 is a mouse RAP nucleic acid sequence. SEQ ID NO:6 is a mouse RAP amino acid sequence encoded by SEQ ID NO:5. Domain 1 can comprise amino acid 1-112 of SEQ ID NO:1 or SEQ ID NO:2. Domain 2 can comprise amino acids 113-215 of SEQ ID NO:1 or SEQ ID NO:2. Domain 3 can comprise amino acids 216-323 of SEQ ID NO:1 or SEQ ID NO:2.

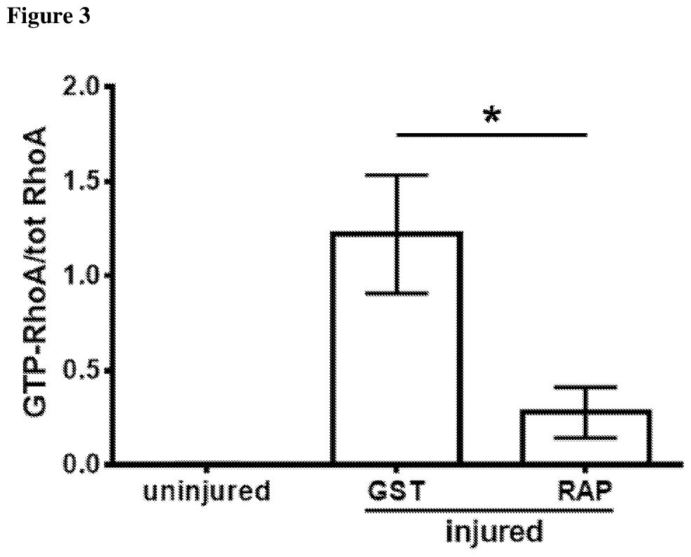

RAP acts as a LRP1 antagonist. It blocks the activation of neuronal RhoA in response to myelin proteins resulting in enhanced neurite outgrowth, which is normally inhibited by myelin. In vivo infusion of RAP into the lesion site of an injured spinal cord results in a significant inhibition of the pathological RhoA activation that occurs post-spinal cord injury, compared to GST, used as a positive control. Spinal cord from uninjured animals is used as a negative control. FIG. 3 shows the increase in RhoA activity after spinal cord injury (GST) and RAP infusion significantly decreases RhoA activity.

RAP also specifically inhibits pathological RhoA activation because of its interaction with LRP1. RAP does not negatively affect endogenous RhoA activation. This reduces the risk of toxicity resulting from off-target ablation of endogenous RhoA function seen in pan-Rho inhibitors, permits greater amounts of a RAP-based therapeutic agent to be used, and is likely to result in greater remyelination over pan-Rho targeting agents.

RAP is also useful for treating CNS disorders because it possesses active blood brain barrier (BBB) transport properties. The bioavailability of RAP to the CNS is substantially greater than agents that do not cross the BBB. Following intravenous RAP infusion, intact RAP is able to still demonstrate linear influx into both skeletal muscle and brain. The amount of intact RAP as a percentage of total injected RAP to enter the brain is approximately 0.9% after 30 minutes. Approximately 70% of RAP that entered the CNS is subsequently detectable in the parenchyma. Intrathecal infusion is approximately 100 times more efficient at delivering RAP to the CNS.

The role of RAP in inhibiting LRP1 and attenuating pathological Rho activation both in vitro and in vivo shows the therapeutic usefulness of RAP in the treatment of MS. As such, the methods and compositions disclosed herein relate to administering RAP, a derivative of RAP, a variant of RAP, or a fragment of RAP to increase, promote, restore, and/or enhance OPC differentiation, e.g., into mature oligodendrocytes; to increase, promote, restore, and/or enhance myelin protein expression; to increase, promote, restore, and/or enhance mature oligodendrocyte marker expression; to increase, promote, restore, and/or enhance myelination; to decrease and/or block pathological RhoA activation in OPC; to alleviate one or more symptoms of MS; and to treat MS. These methods can be readily applied to progressive forms of MS for which there are few viable therapeutic options.

Methods of increasing, promoting, restoring, or enhancing OPC differentiation according to the present disclosure can comprise administering to a subject in need thereof a therapeutically effective amount of one or more of receptor associated protein (RAP), a derivative of RAP, a variant of RAP, or a fragment of RAP. OPC differentiation can be increased, promoted, restored, or enhanced in the presence of myelin debris. Administering can be accomplished intracranially, intrathecally, and/or intravenously. Administering can involve delivery of a pharmaceutical composition that comprises a delivery vehicle and an expression vector that encodes the RAP, derivative of RAP, variant of RAP, or fragment of RAP. The subject can be a mammal, e.g., a human. The derivative of RAP can be a derivative optimized for in vivo delivery to a subject, e.g., optimized for in vivo delivery to a mammal, such as a human. The fragment of RAP can comprise RAP-D3.

The administering step of these methods can be done after a demyelinating event, such as an auto-inflammatory MS episode, and can be done during a MS remission phase and/or a MS acute lesion phase. The methods can alleviate one or more symptoms of MS, including RRMS. The methods can also slow and/or stop progression of MS, for example, progression from RRMS to SPMS.

Methods of increasing, promoting, restoring, or enhancing myelin protein expression according to the present disclosure can comprise administering to a subject in need thereof a therapeutically effective amount of one or more of receptor associated protein (RAP), a derivative of RAP, a variant of RAP, or a fragment of RAP. Administering RAP, a derivative of RAP, a variant of RAP, or a fragment of RAP can also increase and/or enhance myelin protein activity. The myelin protein can be myelin basic protein (MBP). These methods can be done in the presence of myelin debris. Administering can be accomplished intracranially, intrathecally, and/or intravenously. Administering can involve delivery of a pharmaceutical composition that comprises a delivery vehicle and an expression vector that encodes the RAP, derivative of RAP, variant of RAP, or fragment of RAP. The subject can be a mammal, e.g., a human. The derivative of RAP can be a derivative optimized for in vivo delivery to a subject, e.g., optimized for in vivo delivery to a mammal, such as a human. The fragment of RAP can comprise RAP-D3 (e.g., a sequence comprising amino acids 216-323 of SEQ ID NO:1 or SEQ ID NO:2).

The administering step of these methods can be done after a demyelinating event, such as an auto-inflammatory MS episode, and can be done during a MS remission phase and/or a MS acute lesion phase. The methods can alleviate one or more symptoms of MS, including RRMS. The methods can also slow and/or stop progression of MS, for example, progression from RRMS to SPMS.

Methods of increasing, promoting, restoring, or enhancing expression of one or more mature oligodendrocyte markers according to the present disclosure can comprise administering to a subject in need thereof a therapeutically effective amount of one or more of receptor associated protein (RAP), a derivative of RAP, a variant of RAP, or a fragment of RAP. These methods can be done in the presence of myelin debris. Administering can be accomplished intracranially, intrathecally, and/or intravenously. Administering can involve delivery of a pharmaceutical composition that comprises a delivery vehicle and an expression vector that encodes the RAP, derivative of RAP, variant of RAP, or fragment of RAP. The subject can be a mammal, e.g., a human. The derivative of RAP can be a derivative optimized for in vivo delivery to a subject, e.g., optimized for in vivo delivery to a mammal, such as a human. The fragment of RAP can comprise RAP-D3 (e.g., a sequence comprising amino acids 216-323 of SEQ ID NO:1 or SEQ ID NO:2).

The administering step of these methods can be done after a demyelinating event, such as an auto-inflammatory MS episode, and can be done during a MS remission phase and/or a MS acute lesion phase. The methods can alleviate one or more symptoms of MS, including RRMS. The methods can also slow and/or stop progression of MS, for example, progression from RRMS to SPMS.

Methods of increasing, promoting, restoring, or enhancing myelination (e.g., remyelination) according to the present disclosure can comprise administering to a subject in need thereof a therapeutically effective amount of one or more of receptor associated protein (RAP), a derivative of RAP, a variant of RAP, or a fragment of RAP. These methods can be done in the presence of myelin debris. Administering can be accomplished intracranially, intrathecally, and/or intravenously. Administering can involve delivery of a pharmaceutical composition that comprises a delivery vehicle and an expression vector that encodes the RAP, derivative of RAP, variant of RAP, or fragment of RAP. The subject can be a mammal, e.g., a human. The derivative of RAP can be a derivative optimized for in vivo delivery to a subject, e.g., optimized for in vivo delivery to a mammal, such as a human. The fragment of RAP can comprise RAP-D3 (e.g., a sequence comprising amino acids 216-323 of SEQ ID NO:1 or SEQ ID NO:2).

The administering step of these methods can be done after a demyelinating event, such as an auto-inflammatory MS episode, and can be done during a MS remission phase and/or a MS acute lesion phase. The methods can alleviate one or more symptoms of MS, including RRMS. The methods can also slow and/or stop progression of MS, for example, progression from RRMS to SPMS.

Methods of decreasing and/or blocking pathological RhoA activation in an OPC according to the present disclosure can comprise administering to a subject in need thereof a therapeutically effective amount of one or more of receptor associated protein (RAP), a derivative of RAP, a variant of RAP, or a fragment of RAP. These methods can be done in the presence of myelin debris. The methods can be accomplished without negatively inhibiting endogenous RhoA activity. Administering can be accomplished intracranially, intrathecally, and/or intravenously. Administering can involve delivery of a pharmaceutical composition that comprises a delivery vehicle and an expression vector that encodes the RAP, derivative of RAP, variant of RAP, or fragment of RAP. The subject can be a mammal, e.g., a human. The derivative of RAP can be a derivative optimized for in vivo delivery to a subject, e.g., optimized for in vivo delivery to a mammal, such as a human. The fragment of RAP can comprise RAP-D3 (e.g., a sequence comprising amino acids 216-323 of SEQ ID NO:1 or SEQ ID NO:2).

The administering step of such methods can be done after a demyelinating event, such as an auto-inflammatory MS episode, and can be done during a MS remission phase and/or a MS acute lesion phase. The methods can alleviate one or more symptoms of MS, including RRMS. The methods can also slow and/or stop progression of MS, for example, progression from RRMS to SPMS.

Methods of alleviating one or more symptoms of MS according to the present disclosure can comprise administering to a subject in need thereof a therapeutically effective amount of one or more of receptor associated protein (RAP), a derivative of RAP, a variant of RAP, or a fragment of RAP. Such methods can be done in the presence of myelin debris. The method can be accomplished without negatively inhibiting endogenous RhoA activity. Administering can be accomplished intracranially, intrathecally, and/or intravenously. Administering can involve delivery of a pharmaceutical composition that comprises a delivery vehicle and an expression vector that encodes the RAP, derivative of RAP, variant of RAP, or fragment of RAP. The subject can be a mammal, e.g., a human. The derivative of RAP can be a derivative optimized for in vivo delivery to a subject, e.g., optimized for in vivo delivery to a mammal, such as a human. The fragment of RAP can comprise RAP-D3 (e.g., a sequence comprising amino acids 216-323 of SEQ ID NO:1 or SEQ ID NO:2).

The administering step of the methods can be done during a MS remission phase and/or a MS acute lesion phase. The methods can alleviate one or more symptoms of RRMS. The methods can also slow and/or stop progression of MS, for example, progression from RRMS to SPMS.

Methods of treating MS according to the present disclosure can comprise administering to a subject in need thereof a therapeutically effective amount of one or more of receptor associated protein (RAP), a derivative of RAP, a variant of RAP, or a fragment of RAP. The methods can be performed in the presence of myelin debris. Administering can be accomplished intracranially, intrathecally, and/or intravenously. Administering can involve delivery of a pharmaceutical composition that comprises a delivery vehicle and an expression vector that encodes the RAP, derivative of RAP, variant of RAP, or fragment of RAP. The subject can be a mammal, e.g., a human. The derivative of RAP can be a derivative optimized for in vivo delivery to a subject, e.g., optimized for in vivo delivery to a mammal, such as a human. The fragment of RAP can comprise RAP-D3 (e.g., a sequence comprising amino acids 216-323 of SEQ ID NO:1 or SEQ ID NO:2).

The administering step of the methods can be done during a MS remission phase and/or a MS acute lesion phase. The methods can treat RRMS. The methods can also slow and/or stop progression of MS, for example, progression from RRMS to SPMS.

Methods of decreasing and/or blocking LRP1 function in an OPC in the presence of myelin debris according to the present disclosure can comprise administering to a subject in need thereof a therapeutically effective amount of one or more of receptor associated protein (RAP), a derivative of RAP, a variant of RAP, or a fragment of RAP. Administering can be accomplished intracranially, intrathecally, and/or intravenously. Administering can involve delivery of a pharmaceutical composition that comprises a delivery vehicle and an expression vector that encodes the RAP, derivative of RAP, variant of RAP, or fragment of RAP. The subject can be a mammal, e.g., a human. The derivative of RAP can be a derivative optimized for in vivo delivery to a subject, e.g., optimized for in vivo delivery to a mammal, such as a human. The fragment of RAP can comprise RAP-D3 (e.g., a sequence comprising amino acids 216-323 of SEQ ID NO:1 or SEQ ID NO:2).

The administering step of such methods can be done after a demyelinating event, such as an auto-inflammatory MS episode, and can be done during a MS remission phase and/or a MS acute lesion phase. The methods can alleviate one or more symptoms of MS, including RRMS. The methods can also slow and/or stop progression of MS, for example, progression from RRMS to SPMS.

The methods described herein can involve administering a derivative, variant, or fragment of RAP. Suitable derivatives, variants, or fragments can have the same or similar LRP1 antagonism and BBB transport properties of full length RAP. One exemplary derivative is a RAP derivative optimized for in vivo delivery to a subject, e.g., optimized for in vivo delivery to a mammal, such as a human, mouse, or rat. One exemplary fragment can comprise the third domain of RAP (also referred to as domain 3, RAP-D3, or D3, and comprising amino acids 216-323 of SEQ ID NO:1 or SEQ ID NO:2), which binds with nanomolar affinity to LRP1, and by itself is sufficient to reconstitute the antagonistic and BBB transport properties. Fragments comprising the first domain (e.g., a sequence comprising amino acids 1-112 of SEQ ID NO:1 or SEQ ID NO:2) or the second domain of RAP (e.g., a sequence comprising amino acids 113-215 of SEQ ID NO:1 or SEQ ID NO:2) can also be used in the present methods.

A construct comprising one or more RAP-D3 domains can also be used in the methods (i.e., D3 repeats). Constructs comprising one or more of the three RAP domains in various combinations and orders can also be used in the methods, for example, a construct comprising one or more RAP-D1 domains (i.e., D1 repeats), a construct comprising one or more RAP-D2 domains (i.e., D2 repeats), a construct comprising one or more copies of RAP-D1 and RAP-D2 in combination and in various orders (D1-D2; D2-D1; D1-D2-D1; D1-D1-D2; D2-D1-D1; D2-D1-D2; D2-D2-D1; D1-D2-D2; etc.), a construct comprising one or more copies of RAP-D1 and RAP-D3 in combination and in various orders (D1-D3; D3-D1; D1-D3-D1; D1-D1-D3; D3-D1-D1; D3-D1-D3; D3-D3-D1; D1-D3-D3; etc.), and a construct comprising one or more copies of RAP-D2 and RAP-D3 in combination and in various orders (D2-D3; D3-D2; D2-D3-D2; D2-D2-D3; D3-D2-D2; D3-D2-D3; D3-D3-D2; D2-D3-D3; etc.).

Exemplary RAP fragments, derivatives and variants that can be useful in the methods of the present invention also include, but are not limited to, those compounds disclosed in U.S. Pat. Nos. 7,700,554; 7,977,317; 7,569,544; 7,829,537; 8,236,753; 8,440,629; 8,609,103; 8,795,627; 9,062,126; and 7,560,431; in U.S. Pub. Nos. and 2009/0269346; and in International Publication Nos. WO 2008/036682 and WO 2005/002515, the entire contents of each of which is incorporated herein by reference. PCT/US2012/035125 (publication WO 2012/149111 A1) is also incorporated by reference in its entirety.

In the present methods, RAP, the RAP derivative, the RAP variant, or the RAP fragment can be administered to subject intracranially, intravenously, or intrathecally.

The terms “low density lipoprotein receptor-related protein associated protein 1”, “LRPAP1,” “alpha-2-macroglobulin receptor-associated protein,” and “RAP” interchangeably refer to nucleic acids and polypeptide polymorphic variants, alleles, mutants, and interspecies homologs that: (1) have an amino acid sequence that has greater than about 90% amino acid sequence identity, for example, 91%, 92%, 93%, 94%, 95%, 96%, 97%, 98% or 99% or greater amino acid sequence identity, preferably over a region of at least about 25, 50, 100, 200, 300, 400, or more amino acids, or over the full-length, to an amino acid sequence encoded by a RAP nucleic acid (see, e.g., GenBank Accession No. NM 002337.2, SEQ ID NO:3) or to an amino acid sequence of a RAP polypeptide (see, e.g., GenBank Accession No. NP 002328.1, SEQ ID NO:4); (2) bind to antibodies, e.g., polyclonal antibodies, raised against an immunogen comprising an amino acid sequence of a RAP polypeptide (e.g., RAP polypeptides described herein); or an amino acid sequence encoded by a RAP nucleic acid (e.g., RAP polynucleotides described herein), and conservatively modified variants thereof; (3) specifically hybridize under stringent hybridization conditions to an anti-sense strand corresponding to a nucleic acid sequence encoding a RAP protein, and conservatively modified variants thereof; and/or (4) have a nucleic acid sequence that has greater than about 90%, preferably greater than about 91%, 92%, 93%, 94%, 95%, 96%, 97%, 98%, 99%, or higher nucleotide sequence identity, preferably over a region of at least about 25, 50, 100, 200, 500, 1000, 2000 or more nucleotides, or over the full-length, to a RAP nucleic acid (e.g., RAP polynucleotides, as described herein, and RAP polynucleotides that encode RAP polypeptides, as described herein).

The term “subject” as used herein refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of the term “subject.”

As used herein, the terms “alleviating,” “treating,” or “ameliorating” means that one or more clinical signs and/or the symptoms associated with MS or another demyelinating disease are lessened as a result of the actions performed. The signs or symptoms to be monitored will be characteristic of MS and other demyelinating diseases and will be well known to the skilled clinician, as will the methods for monitoring the signs and conditions.

As used herein, the terms “decrease,” “block,” “reduce,” and “inhibit” are used together because it is recognized that, in some cases, a decrease, for example, in Rho activity can be reduced below the level of detection of a particular assay. As such, it may not always be clear whether the activity is “reduced” below a level of detection of an assay, or is completely “inhibited”. Nevertheless, it will be determinable, following a treatment according to the present methods, that the level of Rho activity and/or the level of LRP1 expression in the particular region or cells being tested are at least reduced from the level before treatment.

As used herein, the terms “increase,” “promote,” “restore,” and “enhance” are used together because it is recognized that, in some cases, the quantifiable increase, for example, in OPC differentiation, MBP expression, or myelination activity can be below the level of detection of a particular assay. Similarly, a very low amount of activity can be below the level of detection of a particular assay. As such, it may not always be clear whether the activity is “increased” above a level that is not detectable by an assay, or is “restored” from no activity. Nevertheless, it will be determinable, following a treatment according to the present methods, that the level of activity and/or expression in the particular region or cells being tested is at least increased from the level before treatment.

The term “effective amount” or “therapeutically effective amount” refers to the amount of an active agent sufficient to induce a desired biological result. That result may be alleviation of the signs, symptoms, or causes of a disease, or any other desired alteration of a biological system. The term “therapeutically effective amount” is used herein to denote any amount of the formulation which causes a substantial improvement in a disease condition when applied to the affected areas repeatedly over a period of time. The amount will vary with the condition being treated, the stage of advancement of the condition, and the type and concentration of formulation applied. Appropriate amounts in any given instance will be readily apparent to those skilled in the art or capable of determination by routine experimentation.

A “therapeutic effect,” as used herein, encompasses a therapeutic benefit and/or a prophylactic benefit as described above. A prophylactic effect includes delaying or eliminating the appearance of a disease or condition, delaying or eliminating the onset of symptoms of a disease or condition, slowing, halting, or reversing the progression of a disease or condition, or any combination thereof.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, α-carboxyglutamate, and O-phosphoserine. Amino acid analogs refers to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following seven groups each contain amino acids that are conservative substitutions for one another:

1) Alanine (A), Glycine (G);

2) Aspartic acid (D), Glutamic acid (E);

3) Asparagine (N), Glutamine (Q);

4) Arginine (R), Lysine (K);

5) Isoleucine (I), Leucine (L), Methionine (M), Valine (V);

6) Phenylalanine (F), Tyrosine (Y), Tryptophan (W); and

7) Serine (S), Threonine (T)

A “polynucleotide” is a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. Sizes of polynucleotides are expressed as base pairs (abbreviated “bp”), nucleotides (“nt”), or kilobases (“kb”). Where the context allows, the latter two terms may describe polynucleotides that are single stranded or double-stranded. When the term is applied to double-stranded molecules it is used to denote overall length and will be understood to be equivalent to the term “base pairs”. It will be recognized by those skilled in the art that the two strands of a double stranded polynucleotide may differ slightly in length and that the ends thereof may be staggered as a result of enzymatic cleavage; thus all nucleotides within a double-stranded polynucleotide molecule may not be paired.

The terms “identical” or percent “identity,” in the context of two or more nucleic acids or polypeptide sequences, refer to two or more sequences or subsequences that are the same or have a specified percentage of amino acid residues or nucleotides that are the same (i.e., share at least about 80% identity, for example, at least about 85%, 90%, 91%, 92%, 93%, 94%, 95%, 96%, 97%, 98%, 99% identity over a specified region to a reference sequence, e.g., a RAP polynucleotide or polypeptide sequence, a derivative/variant thereof, or fragment thereof as described herein, when compared and aligned for maximum correspondence over a comparison window, or designated region as measured using one of the following sequence comparison algorithms or by manual alignment and visual inspection. Such sequences are then said to be “substantially identical.” This definition also refers to the compliment of a test sequence. Preferably, the identity exists over a region that is at least about 25 amino acids or nucleotides in length; for example, over a region that is 50-100 amino acids or nucleotides in length, or over the full-length of a reference sequence.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters. For sequence comparison of nucleic acids and proteins to RAP nucleic acids and proteins (and/or derivatives/variants thereof), the BLAST and BLAST 2.0 algorithms and the default parameters discussed below are used.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Nat'l. Acad. Sci. USA 85:2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Ausubel et al., eds., Current Protocols in Molecular Biology (1995 supplement)).

Examples of algorithms that are suitable for determining percent sequence identity and sequence similarity are the BLAST and BLAST 2.0 algorithms, which are described in Altschul et al., J. Mol. Biol. 215:403-410 (1990) and Altschul et al., Nucleic Acids Res. 25:3389-3402 (1977), respectively. Software for performing BLAST analyses is publicly available through the National Center for Biotechnology Information (on the worldwide web at ncbi.nlm.nih.gov/). The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al, supra). These initial neighborhood word hits acts as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

An indication that two nucleic acid sequences or polypeptides are substantially identical is that the polypeptide encoded by the first nucleic acid is immunologically cross reactive with the antibodies raised against the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules or their complements hybridize to each other under stringent conditions, as described below. Yet another indication that two nucleic acid sequences are substantially identical is that the same primers can be used to amplify the sequence.

The terms “administration” or “administering” are defined to include the act of providing a compound or pharmaceutical composition of the invention to a subject in need of treatment. Exemplary acts include providing a compound or pharmaceutical composition intravenously (i.e., intravenous administration), intracranially, (i.e., intracranial administration), and intrathecally (i.e., intrathecal administration). The phrases “systemic administration,” “administered systemically,” “peripheral administration” and “administered peripherally” as used herein mean the administration of a compound, drug or other material other than directly into the central nervous system, such that it enters the subject's system and, thus, is subject to metabolism and other like processes, for example, subcutaneous administration.

Thus, the compounds of the invention can be administered in any way typical of an agent used to treat the particular type of ocular disorder, or under conditions that facilitate contact of the agent with target intraocular cells and, if appropriate, entry into the cells. Entry of a polynucleotide agent into a cell, for example, can be facilitated by incorporating the polynucleotide into a viral vector that can infect the cells. Specific examples of such approaches include, but are not limited to, lenti or adenoviral derived expression systems for RAP or shRNA against LRP1, stable-expressing and secreting cell delivery systems capable of long-term release of RAP (or similar agents such as receptor decoys), or bioavailable topical solutions capable of administration in drop form.

If a viral vector specific for the cell type is not available, the vector can be modified to express a receptor (or ligand) specific for a ligand (or receptor) expressed on the target cell, or can be encapsulated within a liposome, which also can be modified to include such a ligand (or receptor). A peptide agent can be introduced into a cell by various methods, including, for example, by engineering the peptide to contain a protein transduction domain such as the human immunodeficiency virus TAT protein transduction domain, which can facilitate translocation of the peptide into the cell. In addition, there are a variety of biomaterial-based technologies such as nano-cages and pharmacological delivery wafers (such as used in brain cancer chemotherapeutics) which may also be modified to accommodate this technology.

Methods for chemically modifying polynucleotides and polypeptides, for example, to render them less susceptible to degradation by endogenous nucleases or proteases, respectively, or more absorbable through the alimentary tract are well known (see, for example, Blondelle et al., Trends Anal. Chem. 14:83-92, 1995; Ecker and Crook, BioTechnology, 13:351-360, 1995). For example, a peptide agent can be prepared using D-amino acids, or can contain one or more domains based on peptidomimetics, which are organic molecules that mimic the structure of peptide domain; or based on a peptoid such as a vinylogous peptoid. Where the compound is a small organic molecule such as a steroidal alkaloid, it can be administered in a form that releases the active agent at the desired position in the body (e.g., the eye), or by injection into a blood vessel such that the inhibitor circulates to the target cells (e.g., intraocular cells).

The compounds of the invention are also suitably administered by sustained release systems. Suitable examples of sustained-release compositions include, but are not limited to, semi-permeable polymer matrices in the form of shaped articles, e.g., films, or microcapsules. Sustained-release matrices include polylactides (U.S. Pat. No. 3,773,919, EP 58,481 incorporated herein by reference), copolymers of L-glutamic acid and gamma-ethyl-L-glutamate (U. Sidman et al., Biopolymers 22:547-556 (1983)), poly (2-hydroxyethyl methacrylate) (R. Langer et al., J. Biomed Mater. Res. 15:167-277 (1981), and R. Langer, Chem. Tech. 12:98-105 (1982)), ethylene vinyl acetate (R. Langer et al., Id.) or poly-D-(−)-3-hydroxybutyric acid (EP 133,988). Liposomes containing the compounds of the invention may be prepared by methods known in the art: Epstein, et al., Proc. Natl. Acad. Sci. USA 82:3688-3692 (1985); Hwang et al., Proc. Natl. Acad. Sci. USA 77:4030-4034 (1980); EP 52,322; EP 36,676; EP 88,046; EP 143,949; EP 142,641; U.S. Pat. Nos. 4,485,045 and 4,544,545; and EP 102,324. Ordinarily, the liposomes are of the small (about 200-800 Angstroms) unilamellar type in which the lipid content is greater than about 30 mol. percent cholesterol, the selected proportion being adjusted for the optimal delivery of the compounds of the invention.

In certain embodiments, the invention compounds may further be administered (i.e., co-administered) in combination with an anti-inflammatory, antimicrobial, antihistamine, chemotherapeutic agent, antiangiogenic agent, immunomodulator, therapeutic antibody or a neuroprotective agent, to a subject in need of such treatment. Other agents that may be administered in combination with invention compounds include protein therapeutic agents such as cytokines, immunomodulatory agents and antibodies. While not wanting to be limiting, antimicrobial agents include antivirals, antibiotics, antifungals and anti-parasitics. When other therapeutic agents are employed in combination with the inhibitors of the present invention they may be used for example in amounts as noted in the Physician Desk Reference (PDR) or as otherwise determined by one having ordinary skill in the art.

The term “co-administer” and “co-administering” and variants thereof refer to the simultaneous presence of two or more active agents in an individual. The active agents that are co-administered can be concurrently or sequentially delivered. As used herein, RAP can be co-administered with another active agent efficacious in promoting neuronal regeneration in the CNS.

In certain embodiments, the invention compositions may include RAP conjugated neurotrophic or neuroprotective agents. Exemplary neurotrophic or neuroprotective agents include, but are not limited to, neurotrophins (e.g., brain-derived neurotrophic factor (BDNF), nerve growth factor (NGF), neurotrophin-3/4 (NT-3/4), ciliary neurotrophic factor (CNTF)), cyclic nucleotide homologs (e.g., cAMP derivatives), C3 transferase derivatives, stimulators of adenylyl cyclases (e.g., forskolin and other hormones).

In certain embodiments, the compositions for use in the methods of the present invention further comprise a targeting moiety. Targeting moieties include a protein or a peptide which directs localization to a certain part of the body, for example, to the brain or spine, or compartments therein. In certain embodiments, compositions for use in the methods of the present invention are attached or fused to a brain targeting moiety. The brain targeting moieties are attached covalently (e.g., direct, translational fusion, or by chemical linkage either directly or through a spacer molecule, which can be optionally cleavable) or non-covalently attached (e.g., through reversible interactions such as avidin:biotin, protein A:IgG, etc.). In other embodiments, the compounds for use in the methods of the present invention thereof are attached to one more brain targeting moieties. In additional embodiments, the brain targeting moiety is attached to a plurality of compounds for use in the methods of the present invention.

A CNS targeting moiety associated with a compound enhances CNS delivery of such compositions. A number of polypeptides have been described which, when fused to a therapeutic agent, delivers the therapeutic agent through the blood brain barrier (BBB). Nonlimiting examples include the single domain antibody FC5 (Abulrob et al. (2005) J. Neurochem. 95, 1201-1214); mAB 83-14, a monoclonal antibody to the human insulin receptor (Pardridge et al. (1995) Pharmacol. Res. 12, 807-816); the B2, B6 and B8 peptides binding to the human transferrin receptor (hTfR) (Xia et al. (2000) J. Virol. 74, 11359-11366); the OX26 monoclonal antibody to the transferrin receptor (Pardridge et al. (1991) J. Pharmacol. Exp. Ther. 259, 66-70); diptheria toxin conjugates. (see, for e.g., Gaillard et al., International Congress Series 1277:185-198 (2005); and SEQ ID NOs: 1-18 of U.S. Pat. No. 6,306,365. The contents of the above references are incorporated herein by reference in their entirety).

Accordingly, in another aspect, the methods of the invention are useful for providing a means for practicing personalized medicine, wherein treatment is tailored to a subject based on the particular characteristics of the disorder from which the subject is suffering. The method can be practiced, for example, by contacting a sample of cells from the subject with at least one inhibitor of LRP1 expression or activity, wherein a decrease in LRP1 expression or activity in the presence of the inhibitor as compared to the LRP1 expression or activity in the absence of the inhibitor identifies the inhibitor as useful for treating the disease. The sample of cells examined according to the present method can be obtained from the subject to be treated, or can be cells of an established disease cell line or known disease of the same type as that of the subject. In one aspect, the established cell line can be one of a panel of such cell lines, wherein the panel can include different cell lines of the same type of disease and/or different cell lines of different diseases associated with demyelination. Such a panel of cell lines can be useful, for example, to practice the present method when only a small number of cells can be obtained from the subject to be treated, thus providing a surrogate sample of the subject's cells, and also can be useful to include as control samples in practicing the present methods.

As used herein, the terms “sample” and “biological sample” refer to any sample suitable for the methods provided by the present invention. In one embodiment, the biological sample of the present invention is a tissue sample, e.g., a biopsy specimen such as samples from needle biopsy. In other embodiments, the biological sample of the present invention is a sample of bodily fluid, e.g., intraocular fluid, serum, and plasma.

As used herein “corresponding normal cells” means cells that are from the same organ and of the same type as the cells being examined. In one aspect, the corresponding normal cells comprise a sample of cells obtained from a healthy individual. Such corresponding normal cells can, but need not be, from an individual that is age-matched and/or of the same sex as the individual providing the cells being examined. In another aspect, the corresponding normal cells comprise a sample of cells obtained from an otherwise healthy portion of tissue of a subject having demyelination disorder.

Once disease is established and a treatment protocol is initiated, the methods of the invention may be repeated on a regular basis to evaluate whether the level of LRP1 expression or activity in the subject begins to approximate that which is observed in a normal subject. Alternatively, or in addition thereto, the methods of the invention may be repeated on a regular basis to evaluate whether symptoms associated with the particular disease from which the subject suffers have been decreased or ameliorated. The results obtained from successive assays may be used to show the efficacy of treatment over a period ranging from several days to months to years. Accordingly, the invention is also directed to methods for monitoring a therapeutic regimen for treating a subject having disease resulting in demyelination. A comparison of the levels of LRP1 expression or activity and/or a comparison of the symptoms associated with the particular ocular disorder prior to and during therapy indicates the efficacy of the therapy. Therefore, one skilled in the art will be able to recognize and adjust the therapeutic approach as needed.

The total amount of a compound or composition to be administered in practicing a method of the invention can be administered to a subject as a single dose, either as a bolus or by infusion over a relatively short period of time, or can be administered using a fractionated treatment protocol, in which multiple doses are administered over a prolonged period of time. One skilled in the art would know that the amount of the inhibitor of LRP1 expression or activity to treat ocular disorders in a subject depends on many factors including the age and general health of the subject as well as the route of administration and the number of treatments to be administered. In view of these factors, the skilled artisan would adjust the particular dose as necessary.

The present methods can involve administering RAP via intracranial, intrathecal, or intravenous infusion. While intravenous infusion is generally considered a favorable route of administration, intrathecal infusion is a clinically relevant and direct mode of delivery for a drug capable of enhancing remyelination. As RAP is actively transported across the blood brain barrier (BBB) and into the CNS in vivo, these routes of administration can be used in the present methods.

### EXAMPLES

Having now described the present methods and compositions in detail, the same will be more clearly understood by reference to the following examples, which are included herein for purposes of illustration only and are not intended to be limiting of the disclosure.

### Example 1: RAP Derivative Increases OPC Differentiation in a Dose Dependent Manner

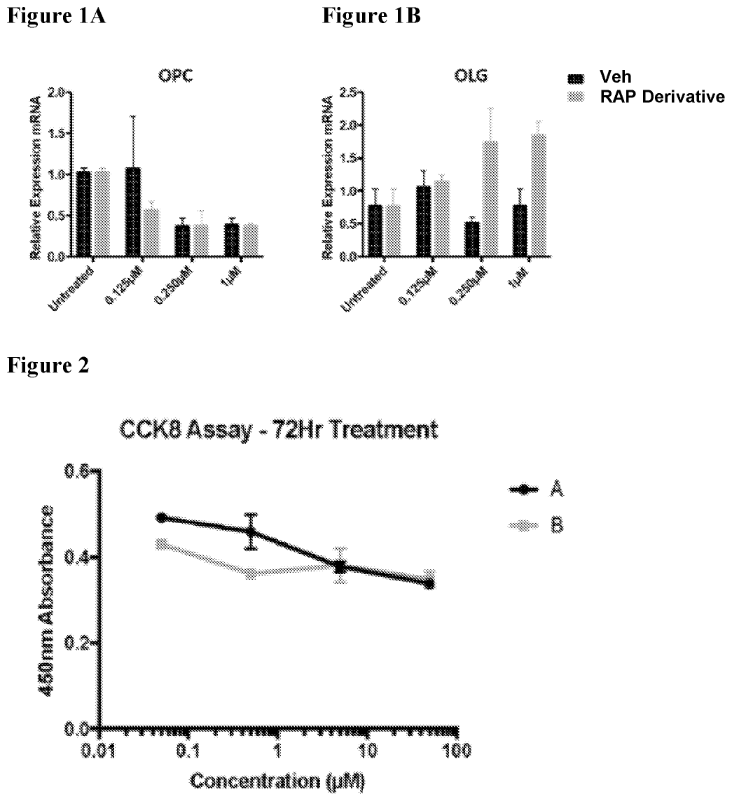

A RAP derivative optimized for in vivo delivery is used to test the treatment of mouse OPC with RAP. OPC are cultured in non-differentiating or differentiating media supplemented with T3. The cell cultures are treated with increasing doses of the RAP derivative or vehicle (PBS) control for 48 hours (untreated, 0.125 μM, 0.250 μM, and 1 μM). Cells are then assessed for markers of OPC differentiation, including mRNA levels of MBP. FIG. 1 shows the mRNA expression levels with the left bar of each pair representing the vehicle and the right bar of each pair representing the RAP derivative.

No increase in MBP expression is observed in non-differentiating media (labeled OPC in FIG. 1). In the differentiating media (labeled OLG in FIG. 1), treatment of OPC with the RAP derivative results in a dose-dependent enhancement of OPC maturation and myelin expression in vitro. A similar effect in vivo and in other mammalian species is expected from these data.

Expression of MBP is also an indicator of the extent of myelination. Thus, the increase in MBP expression from the RAP derivative administration also indicates increased and/or enhanced myelination. This is the result of the RAP derivative acting as a LRP1 antagonist and blocking pathological RhoA activation. By promoting and enhancing OPC maturation and myelination, administering the RAP derivative will alleviate symptoms of MS, slow or prevent MS progression, and/or treat MS.

### Example 2: Cellular Tolerance of RAP Derivative

The effect of the RAP derivative optimized for in vivo delivery on mouse OPC viability is tested at multiple doses, up to 50 μm (50 times the maximum dose rate). OPC viability, relative to a vehicle, is unaffected after 72 hours of treatment at the maximum dose. This indicates that the RAP derivative is well-tolerated by OPC and controls for any cellular differentiation under culture conditions caused by cellular stress. The CCK-8 colorimetric analysis is shown in FIG. 2 (RAP derivative identified by line A and vehicle control identified by line B). OPC in vivo and in other mammalian species are expected to also be tolerant to RAP derivatives and RAP based on these results.

### Example 3: RAP Blocks RhoA Activation after Spinal Cord Injury

The effect of RAP on RhoA activation following spinal cord injury is assessed in rats. RAP or GST (vehicle, as a positive control) are infused into the injury site for five days after injury prior to analyzing RhoA activity. Utilizing intrathecal infusion, a solution of 10 μM RAP is infused by osmotic pump at 1 μl/hr. Uninjured animals are used as a negative control. The results are shown in FIG. 3. Spinal cord injury increases RhoA activity over the uninjured animals. RAP infusion significantly decreases RhoA activity, when compared to GST (p<0.05).

It will be readily apparent to one of ordinary skill in the relevant arts that other suitable modifications and adaptations to the methods and compositions described herein can be made with departing from the scope of the disclosure or any embodiment thereof.

1. A method of increasing, promoting, restoring, or enhancing OPC differentiation comprising administering to a subject in need thereof a therapeutically effective amount of one or more of receptor associated protein (RAP), a derivative of RAP, a variant of RAP, or a fragment of RAP.

2-101. (canceled)


SEQUENCE LISTING (6 sequences)

SEQ ID NO: 1 - protein, 323 aa, Rattus norvegicus
YSREKNEPEMAAKRESGEEFRMEKLNQLWEKAKRLHLSPVRLAELHSDLKIQERDELNWKKLKVEGLDGDGEKEAKLVHNLNVILARYGLDGRKDTQTVHSNALNEDTQDELGDPRLEKLWHKAKTSGKFSSEELDKLWREFLHYKEKIHEYNVLLDTLSRAEEGYENLLSPSDMTHIKSDTLASKHSELKDRLRSINQGLDRLRKVSHQGYGPATEFEEPRVIDLWDLAQSANFTEKELESFREELKHFEAKIEKHNHYQKQLEISHQKLKHVESIGDPEHISRNKEKYVLLEEKTKELGYKVKKHLQDLSSRVSRARHNEL

SEQ ID NO: 2 - protein, 323 aa, Homo sapiens
YSREKNQPKPSPKRESGEEFRMEKLNQLWEKAQRLHLPPVRLAELHADLKIQERDELAWKKLKLDGLDEDGEKEARLIRNLNVILAKYGLDGKKDARQVTSNSLSGTQEDGLDDPRLEKLWHKAKTSGKFSGEELDKLWREFLHHKEKVHEYNVLLETLSRTEEIHENVISPSDLSDIKGSVLHSRHTELKEKLRSINQGLDRLRRVSHQGYSTEAEFEEPRVIDLWDLAQSANLTDKELEAFREELKHFEAKIEKHNHYQKQLEIAHEKLRHAESVGDGERVSRSREKHALLEGRTKELGYTVKKHLQDLSGRISRARHNEL

SEQ ID NO: 3 - DNA, 1586 bp, Homo sapiens
agaccgcgcggtgggtgggggcggggtagtgggcggggcatcggcaggctaggttttctccgcgcagcgccagtcgcagaggaagatggcgccgcggagggtcaggtcgtttctgcgcgggctcccggcgctgctactgctgctgctcttcctcgggccctggcccgctgcgagccacggcggcaagtactcgcgggagaagaaccagcccaagccgtccccgaaacgcgagtccggagaggagttccgcatggagaagttgaaccagctgtgggagaaggcccagcgactgcatcttcctcccgtgaggctggccgagctccacgctgatctgaagatacaggagagggacgaactcgcctggaagaaactaaagcttgacggcttggacgaagatggggagaaggaagcgagactcatacgcaacctcaatgtcatcttggccaagtatggtctggacggaaagaaggacgctcggcaggtgaccagcaactccctcagtggcacccaggaagacgggctggatgaccccaggctggaaaagctgtggcacaaggcgaagacctctgggaaattctccggcgaagaactggacaagctctggcgggagttcctgcatcacaaagagaaagttcacgagtacaacgtcctgctggagaccctgagcaggaccgaagaaatccacgagaacgtcattagcccctcggacctgagcgacatcaagggcagcgtcctgcacagcaggcacacggagctgaaggagaagctgcgcagcatcaaccagggcctggaccgcctgcgcagggtcagccaccagggctacagcactgaggctgagttcgaggagcccagggtgattgacctgtgggacctggcgcagtccgccaacctcacggacaaggagctggaggcgttccgggaggagctcaagcacttcgaagccaaaatcgagaagcacaaccactaccagaagcagctggagattgcgcacgagaagctgaggcacgcagagagcgtgggcgacggcgagcgtgtgagccgcagccgcgagaagcacgccctgctggaggggcggaccaaggagctgggctacacggtgaagaagcatctgcaggacctgtccggcaggatctccagagctcggcacaacgaactctgaaggcattggggagcccagcccggcagggaagaggccagcgtgaaggacctgggctcttggccgtggcatttccgtggacagcccgccgtcagggtggctggggctggcacgggtgtcgaggcaggaaggattgtttctggtgactgcagccgctgccgtcgcgacacagggcttggtggtggtagcatttgggtctgagatcggcccagctctgactgaaggggcttggcttccactcagcatcagcgtggcagtcaccaccccagtgaggacctcgatgtccagctgctgtcaggtctgatagtcctctgctaaaacaacacgatttacataaaaaatcttacacatctgccaccggaaataccatgcacagagtccttaaaaaatagagtgcagtatttaaaccacccgaaaaaaaaaaaaaaaa

SEQ ID NO: 4 - protein, 357 aa, Homo sapiens
MAPRRVRSFLRGLPALLLLLLFLGPWPAASHGGKYSREKNQPKPSPKRESGEEFRMEKLNQLWEKAQRLHLPPVRLAELHADLKIQERDELAWKKLKLDGLDEDGEKEARLIRNLNVILAKYGLDGKKDARQVTSNSLSGTQEDGLDDPRLEKLWHKAKTSGKFSGEELDKLWREFLHHKEKVHEYNVLLETLSRTEEIHENVISPSDLSDIKGSVLHSRHTELKEKLRSINQGLDRLRRVSHQGYSTEAEFEEPRVIDLWDLAQSANLTDKELEAFREELKHFEAKIEKHNHYQKQLEIAHEKLRHAESVGDGERVSRSREKHALLEGRTKELGYTVKKHLQDLSGRISRARHNEL

SEQ ID NO: 5 - DNA, 2922 bp, Mus musculus
aggaagatggcgcctcgaagagagagggtctctacgctgccccggctccaactgctagtgctgttgttgctgccgctgatgcttgtgccccagcccatagcaggccatggcggcaagtactcgcgagagaagaacgagccggagatggccgccaagcgcgagtccggggaggagttccgcatggagaagctgaaccagctatgggagaaggccaagcggcttcatctgtctcctgtgaggctggccgagctgcattctgacctgaagatacaagagagggatgaactcaactggaaaaagctgaaggtggaaggcttggataaggatggggagaaagaagcaaaactgatccacaacctcaacgtcatcctggccagatacggactggatgggaggaaggacgcccagatggtgcacagcaacgccctcaatgaagacacccaggatgagctgggggaccccaggctggaaaagctgtggcacaaggcaaagacatcagggaaattctccagtgaagagctggacaagctgtggagagagtttctgcattacaaagagaagatccaggagtacaatgtgctgctagacacactgagcagagctgaagaaggttatgagaaccttctcagtccctcggacatggcccacatcaagagcgacaccctgatcagcaagcacagtgagctgaaggacagactgcgcagtatcaaccagggcttggaccgcctgcggaaggtcagccaccagggctatggctccaccactgagtttgaagagccccgggtgatagatctgtgggacctggctcagtctgccaacttcactgagaaggaactggagtcattcagggaggagctcaagcactttgaggccaaaattgaaaagcacaaccactaccagaagcagctggagatttcccaccagaagctgaagcacgtggagagcatcggcgaccccgagcacatcagccgcaacaaggagaaatacgtgctgctggaggagaagaccaaggagctgggctacaaggtgaagaagcatctacaggacctgtctagcagggtctcaagggctcggcacaatgagctctgaggaccagaagccaccagcagcagcctagagagaacacttgaagacaccgggagctgtcagcatgtcatcggcttgcatagacctgaggtgactggtgtggctgaccaccgtggcaaggaggatcccttgaaataccaagctgatcctacagtggctggcaaggacttattttctttcaagcaagtgtagttgtcaccaccctggacgagggccttgggtaccgctaccagtgagataggactggactccgagctgcagcacaacagtttatattgaaatcacataaacctgcctgccactggaaacattctgtacagagtccttaaatacatggcagagttttgagccctcgagtccttgcctggtatctcgaaggatgggtccattgagccatggcaaattaagttacagatgaagggggaaggcgaagactagtctacccactggaagcatttccctccctgcttcacgcaccctctccctctctggggatctggccccagacagttttaaatcaagagatttcaggacaaagatggcagaggtgaaagcctgcaggtcttgcagggcgaacgttttctggccccctccacccccaaagaccctgatcagcttccagatccagtctctgttccctcacacagaatagccaaaattttgtctgaccccagcagctgatcctggaagatcaggccattgagtttgttagccgctgaatctcagcccagaaattggaatctgcctatcaggggtgcgcagttaaccatgtttaagaatatcacagccggaaagaggctcagtgagtaaaatgttcatacagcaagcttgaggtctacagtttggatctgcagagccttgctgtgtttacttacctgcagtcccagcacttgagaggcagagaaagtttatcccagccaaataaacaaaggtggagtatatgggagaaggcactctcccactctctgatgtgtgcatacacttacatgtgtacctgtacacaggtgaccactcatagatactcatatatgggtgaaactcccagggcatcatcgtgcaagaagcccatgggttaaggacagcagccgcaaggaacagtaggcagtgtggttccctcacccatgctgtgtctgccaccatccaggccagctgggatgtgtccctctgagcactagtagatatcttcccagaaccctcagcagtgtctgaggctcagagaaattcttgttgctagggtctcagctctgcctttctggttcagcccaacaccaaagcctcctgtgcctcacagcccaggggactcaggctcagagtcaactgctgggggagtgcctggctgtcagatgcaccttcactaatgtttgtctccatccctagagccagaggccatggctgcatgctaaggcttagaaatgggcacgttgaagtaacatagcagtatacagcccagtgaaccatctcagagtgagtgatagagagcccacagctatgttagggagaggtgtgacctgtgtgggagctgcctgcttctggagcagatctctagtcaggaggccactcctgactaagctctcagatatcagatcctgcaaacttccctgggtgagtgtgactcccaccgcaagggaacctcaagctacaaacttagatgcggttctggctagcaggctctgccacctagcaacagtgactgaaagtcctcaggctgacaggtcaccagtcacattatggtaaaggagaacaggttcatgcaagttgtcctctcactatgcgtgctacggcacatgggttcacacaatgaataaaatatcttttaaagcaaaaaaaaaaaaaaaa

SEQ ID NO: 6 - protein, 360 aa, Mus musculus
MAPRRERVSTLPRLQLLVLLLLPLMLVPQPIAGHGGKYSREKNEPEMAAKRESGEEFRMEKLNQLWEKAKRLHLSPVRLAELHSDLKIQERDELNWKKLKVEGLDKDGEKEAKLIHNLNVILARYGLDGRKDAQMVHSNALNEDTQDELGDPRLEKLWHKAKTSGKFSSEELDKLWREFLHYKEKIQEYNVLLDTLSRAEEGYENLLSPSDMAHIKSDTLISKHSELKDRLRSINQGLDRLRKVSHQGYGSTTEFEEPRVIDLWDLAQSANFTEKELESFREELKHFEAKIEKHNHYQKQLEISHQKLKHVESIGDPEHISRNKEKYVLLEEKTKELGYKVKKHLQDLSSRVSRARHNEL